(19) United States
(12) Patent Application Publication
(10) Pub. No.: US 2014/0316326 A1
(43) Pub. Date: Oct. 23, 2014

(54) DEVICE FOR THE TREATMENT OF AN OCULAR DISEASE

(71) Applicant: Francine Behar-Cohen, Paris (FR)

(72) Inventor: Francine Behar-Cohen, Paris (FR)

(73) Assignee: Institut National de la Sante et de la Recherche Medicale (INSERM), Paris (FR)

(21) Appl. No.: 14/238,911

(22) PCT Filed: Aug. 14, 2012
(86) PCT No.: PCT/IB2012/054145

§ 371 Date: Jul. 3, 2014

(30) Foreign Application Priority Data
Aug. 16, 2011 (EP) 11177638.1

(51) Int. Cl. A61F 9/00

(52) U.S. Cl. CPC A61F 9/0026
USPC 604/21

(57) ABSTRACT

The present invention relates to an injection device comprising: —a first support (14) having a cup-shaped first contact surface (18) intented to come into contact with a first region of an outside surface of an eye, —a set of at least four injection needles (17) in fluid communication with each other and protruding from said first contact surface (18) at respective insertion points (22) so that the distance between the distal end (26) of any of said injection needles to said first contact surface is between 0.6 mm and 1.3 mm, the insertion points of the injection needles on the first contact surface being spread on said first contact surface so that the diameter (D′) of the largest circle (C′) that it is possible to include completely in the convex surface (E) defined by said insertion points on a front view of said first contact surface, without any insertion point being included in said circle, is less than 8 mm.

### FIELD OF THE INVENTION

The present invention relates to an injection device for the treatment of an ocular disease in a subject.

### BACKGROUND OF THE INVENTION

In recent years, there have been exciting new advances for the treatment of ocular diseases such as age-related macular degeneration and diabetic retinopathy, using biotherapies. Because the eye is a small, confined organ, isolated by barriers, it has been identified as an organ of choice for local gene therapy.

For example, hereditary retinal dystrophies are due to mutations in gene encoding proteins in photoreceptors (cones and rods) or in retinal pigment epithelial cells (RPE). Whilst gene replacement in photoreceptor cells is still under pre-clinical evaluation, the most striking advances in this field have been made for RPE65 gene replacement in RPE cells, for the treatment of Leber congenital amaurosis (LCA). Not only was it shown that viral gene transfer in the RPE was feasible and efficient in animal models, but recently, patients have received the sub retinal injection of rAAV4 with promising functional results, providing hope for patients suffering from blinding diseases.

Viral vectors allow efficient transfection of RPE cells and have served to validate proof of concepts, but the long-term persistence of viral particles into the retina and the brain continues to raise safety concerns, particularly when treatment is being applied in young children.

When injected into the vitreous, viral vectors do not reach the RPE cells and only their sub-retinal injection have been shown effective for targeting RPE cells or photoreceptors. Moreover, using the sub retinal injection, RPE cells are only transfected in, and in the vicinity of the detached retina area, which implies detaching the macula when central vision recovery is targeted. Such a macular detachment may be associated with vision threatening. Indeed, it is well known that poor vision recovery after retinal detachment is correlated with macular detachment. Recent work using spectral domain OCT has provided evidence that following successful surgical treatment of retinal detachment, 62% of the eyes presented anatomical foveal abnormalities and that particularly, external limiting membrane disruption, observed only when the macula was detached before surgery, was associated with the worst vision prognosis. Even if controversies still exist regarding the factors that may predict vision recovery after macular detachment, the health of the macula at the time of reattachment is probably the most critical variable. In diseased eyes, knowing the uncertainty of central vision recovery after macular detachment, it is difficult to ensure that submacular injection is not risky.

Many non-viral gene transfer vectors or methods have been developed and adapted for ocular gene therapy (Andrieu-Soler C Mol Vis 2006 12:1334; Bejjani R A Sury Ophthalmol 2007 52:196; Bloquel C Adv Drug Deliv Rev 2006 58:1224). Among those, electroporation, also called “electrotansfer” where the current drives plasmid DNA into cells, is among the most efficient ((Mir L M Adv Genet 2005 54:83; Mir L M Methods Mol Biol 2008 423:3; Isaka Y Expert Opin Drug Deliv 2007 4:561) and has been developed up to clinical evaluation (Daud A I J Clin Oncol 2008 26:5896). Previous reports have shown that after sub retinal administration of the plasmids, electroporation allowed the efficient tranfection of new-born murine RPE (Matsuda T Proc Natl Acad Sci USA 2004 101:16) and delayed retinal degeneration in animal models (Chen B Science 2009 323:256). Efficient and prolonged RPE transfection was also achieved in the adult rat using a combination of sub retinal plasmids injection containing specific RPE promoter and electroporation (Kachi S Gene Ther 2005 12:843; Johnson C J Mol Vis 2008 14:2211).

The suprachoroidal space is a potential space in the eye that is located between the choroid, which is the inner vascular tunic, and the sclera, the outer layer of the eye. The suprachoroidal space extends from the anterior portion of the eye posterior to the ciliary body to the posterior portion of the eye up to the optic nerve. The suprachoroidal space of the eye has been thus studied as a possible route for drug delivery. See, e.g., Olsen, et al., American J. Opthamology 142(5): 777-87 (November 2006); PCT Patent Application Publication No. WO 2007/100745 to Iscience Interventional Corporation. The suprachoroidal space may indeed provide a potential route of access from the anterior region of the eye to treat the posterior region. However said route has not been envisaged for non-viral gene therapy.

WO 2006/123248 describes a device for administering a composition by electroporation. In the embodiment shown in FIG. 16, the device comprises first and second annular electrodes, which are separately placed on the surface of the eye, in a concentric configuration. One of the electrodes comprises several parallel injection needles. The placement of the electrodes on the surface of the eye is awkward. In addition, the positioning is relatively imprecise. Finally, these electrodes are not designed for an electroporation into the suprachoroidal space.

WO 2007/131050 describes a device for administrating a composition in a patient's eye. This device may include an array of microneedles.

There is a need for an efficient electroporation device which may be used to introduce a pharmaceutical composition into the suprachoroidal space and then transfer an agent contained in said pharmaceutical composition into ocular cells.

It is an object of the invention to provide such a device.

### SUMMARY OF THE INVENTION

To this end, the invention proposes an injection device comprising:
a first support having a cup shaped first contact surface intended to come into contact with a first region of an outside surface of an eye, posterior to the limbus of said eye, preferably so as to match said first region,
a set of at least four injection needles in fluid communication with each other and protruding from said first contact surface at respective insertion points so that the distance between the distal end of any of said injection needles to said first contact surface is between 0.6 mm and 1.3 mm,
   the insertion points of the injection needles on the first contact surface being spread on said first contact surface so that the diameter of the largest circle that it is possible to include completely in the convex surface defined by said insertion points on a front view of said first contact surface, without any insertion point being included in said circle, is less than 12 mm.

As will be described in more detail later in the description, the inventors have discovered that such an injection device is very efficient for electroporation in the suprachoroidal space, in particular because it provides a plurality of injection needles spread on the first contact surface, allowing an homogenous distribution of the pharmaceutical composition, in particular containing a plasmid DNA, in the suprachoroidal space without the induction of a large detachment area.

The shape of the first contact surface also provides precision in the positioning of the distal ends of the injection needles, so that the injection may be precisely placed between the choroid and the sclera layers, which are normally in contact with each other.

Moreover, the plurality of injection needles and their repartition avoid applying high pressures to make the injected pharmaceutical composition progress between the two layers.

Finally, the plurality of injection needles and their repartition enable desirable diffusion of the injected composition within the suprachoroidal space.

Preferably, the set of injection needles and/or the first contact surface are configured to be used as a first electrode for an electroporation device. The electroporation may therefore be the most efficient precisely in the area where the composition was injected.

Preferably, an injection device according to the invention comprises one or more of the following optional characteristics:
Preferably, the diameter of the largest circle that it is possible to include completely in the convex surface defined by said insertion points on a front view of said first contact surface, without any insertion point being included in said circle, is less than 10 mm, than 8 mm, than 6 mm, less than 5 mm, less than 4 mm, less than 3 mm, less than 2 mm, less than 1 mm, less than 0.5 mm;
Preferably, the diameter of the largest circle that it is possible to include completely in the surface defined by the outer contour of said first contact surface on a front view, without any insertion point being included in said circle, is less than 12 mm, less than 10 mm, less than 8 mm, or less than 6 mm;
Any injection needle is spaced from an adjacent injection needle by a distance greater than 1 mm, or greater than 2 mm, or greater than 3 mm, and/or less than 8 mm, less than 6 mm, less than 5 mm, or less than 4 mm. Put differently, for any injection needle under consideration, it is possible to find another injection needle which is spaced from said injection needle under consideration by said distance;
The insertion points are spread homogeneously in said convex surface on said front view; in an embodiment, the insertion points are spread homogeneously in the surface defined by said outer contour on said front view;
The device preferably comprises more than 5, more than 7, more than 10, more than 15, more than 20, more than 25 and/or less than 50, less than 40 or less than 30 injection needles, these injection needles being different or, preferably, identical;
The distance between the first contact surface and the distal end of any injection needle is preferably more than 0.7 mm, more than 0.8 mm and/or less than 1.2 mm, less than 1.1 mm, or less than 1.0 mm, or less than 0.9 mm, so as to limit the length by which the injection needles may be inserted into the eye;
Said injection needles are designed in such a way that their respective distal ends reach the suprachoroidal space of an eye when the first contact surface is in contact with the outside surface of a human adult eye;
At least one, and preferably all injection needles have a respective proximal end rigidly fixed on said first support (that is to say permanently immobilized on the first support);
The shape of said first contact surface is spheroidal or ellipsoidal, preferably so as to correspond to the shape of the anterior or posterior part of the outside surface of an eye;
The radius of curvature at any point of the first contact surface is greater than 9 mm, greater than 10 mm or greater than 11 mm, and/or less than 15 mm, less than 14 mm, less than 13 mm, or less than 12 mm;
The first contact surface has the form of a spheroidal or ellipsoidal band, in particular extending along an envelope surface corresponding to the shape of the anterior or posterior part of the outside surface of an eye;
The first contact surface has a surface area of greater than 30 mm², greater than 40 mm², greater than 50 mm², greater than 60 mm², greater than 80 mm², greater than 100 mm², greater than 150 mm², greater than 200 mm², and/or less than 900 mm², less than 800 mm², less than 700 mm², less than 600 mm², or less than 500 mm²;
The injection device is provided with a locating mark following an arc of a circle configured so that an operator may position said locating mark in contact with the limbus of an eye;
The arc of a circle of the locating mark has a radius of greater than 5 mm, greater than 6 mm, and/or of less than 8 mm or less than 7 mm;
The injection needles are configured so that, when the locating mark is positioned bearing on the limbus, no injection needle may penetrate into the outside surface of the eye at a distance less than 4 mm, preferably less than 5 mm away from the limbus; all the insertion points of the injection needles are preferably more than 4 mm, preferably more than 5 mm away from the locating mark;
The injection device comprises a first proximal part, the first support being rotationally mounted on said first proximal part.
The injection needles substantially extend along a common general direction, the first support being rotationally mounted on said first proximal part around an axis substantially perpendicular to said direction, the axis of rotation being preferably substantially perpendicular to the general direction of the injection needles;
The injection device comprises a first electrode designed to be electrically connected to a first terminal of an electrical generator;
The first electrode comprises one, preferably several, preferably all the injection needles, and/or at least a part, preferably the whole region of the first support defining the first contact surface;
The first contact surface is, at least partially, preferably completely, defined by an electrically conductive material;
The injection device comprises an electrical first connector, making it possible to electrically connect said first electrode to said first terminal of an electrical generator;
The injection device comprises a second electrode designed to be electrically connected to a second terminal of said electrical generator and mobile relative to the first electrode between a close position and a remote position in which the second electrode is close to and remote from the first electrode, respectively, the second electrode being guided during the movement between the remote position and the close position;
The injection device comprises first and second arms supporting said first and second electrodes, respectively, the movement of the second arm being guided relative to the first arm, preferably the second arm being rotationally mounted on the first arm, in particular like two arms of a pair of scissors;
The injection device comprises elastic means, for instance a spring, configured to force the second electrode toward the close position;
The second electrode comprises an electrically conductive second contact surface, preferably having a shape and/or dimensions similar to those of the first contact surface, the second contact surface being configured to be electrically connected to a second terminal of an electrical generator;
The second contact surface is preferably cup-shaped and configured so as to be in contact, in the close position, with a second region of said outside surface of the eye, preferably so as to match said second region of said outside surface, the second region being opposite to the first region (relative to the centre of the eye), in particular when the locating mark is bearing on the limbus of the eye.

### Electroporation Device

The invention also relates to an electroporation device comprising
an injection device according to the invention, and
an electrical generator,
   the set of injection needles and/or the first contact surface, forming a first electrode, being electrically connected to one and the same terminal of the electrical generator, or “first terminal”.

Preferably, the injection device comprises a second electrode and the electrical generator is electrically connected to said first and second electrodes so as to be able to generate an electrical field between said first and second electrodes. Preferably, the electrical generator is designed to promote the electroporation of a composition injected into an eye by means of the injection needles of the injection device.

### Method

The invention also relates to a method for injecting a composition into the suprachoroidal space of an eye by means of an electroporation device according to the invention, said method comprising the following steps:
a) inserting the injection needles into the eye until the first contact surface comes into contact with a first region of the outside surface of the eye, the injection needles being configured so that, in this position, they open out into said suprachoroidal space,
b) injecting said composition through said injection needles,
c) independently of the preceding steps, applying a counter electrode, preferably a second electrode of the injection device, on a second region of the outside surface of the eye, the second region being substantially opposite to the first region relatively to the centre of the eye,
d) independently of the preceding steps, connecting the first electrode and the counter electrode to said first and second terminals, respectively,
e) generating an electrical field between said first and counter electrodes with said electrical generator, the electrical field being adapted to promote electroporation.

Before step a), the method preferably comprises a step in which a locating mark is placed in contact with the limbus of the eye.

Preferably, step a) comprises rotating the first support on the first proximal part, between disengaged and engaged positions in which the injection needles are extending outside the eye and, at least partially, outside the eye, respectively.

### BRIEF DESCRIPTION OF THE FIGURES

Other features and advantages of the invention will become clear upon reading the following detailed description and by examining the attached drawing, in which:

FIG. 1 shows an electroporation device according to the invention;

FIG. 2 shows a perspective view of a detail of an injection device in a preferred embodiment of the invention;

FIG. 3 shows a front view of the cup 16 of the injection device shown in FIG. 2;

FIG. 4 shows, along the transverse plane B shown in FIG. 3, a partial cross section of the device shown in FIG. 2, and

FIG. 5 represents a particular embodiment of the first contact surface.

In the various figures, identical reference signs are used to designate identical or similar elements.

### DEFINITIONS

“First” and “second” are used to distinguish corresponding elements, but do not limitate the invention. In particular, an injection device according to the invention may comprise only one electrode.

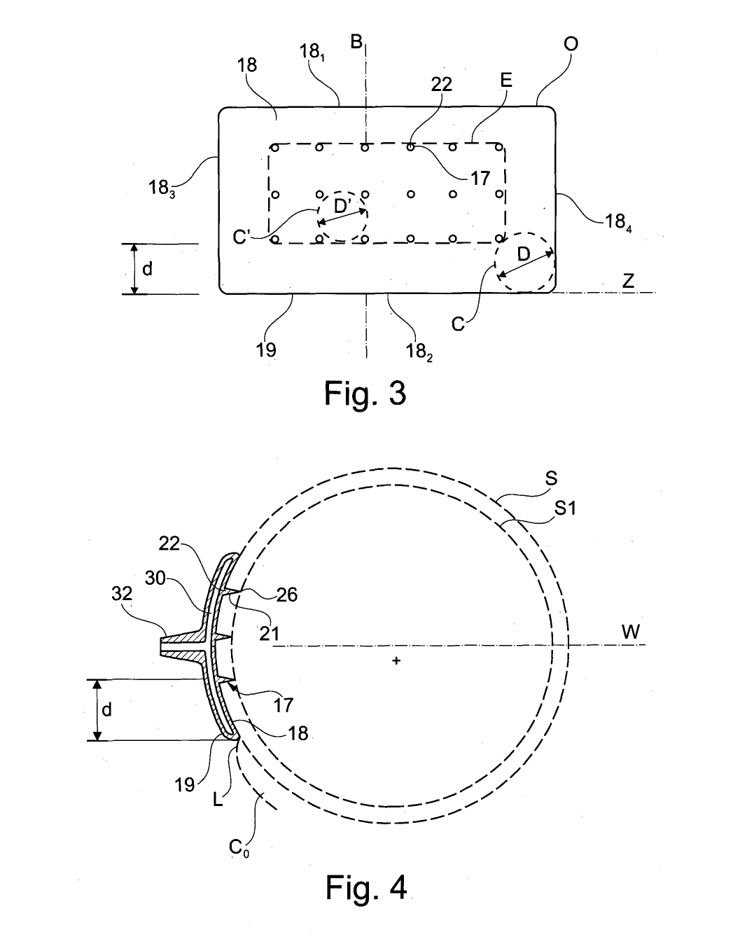

The “convex surface” defined by the insertion points of the needles is the surface of the convex envelope of these insertion points on a front view of the first contact surface. The “convex envelope” is the convex (as view from outside said envelope), closed line, having a minimum length and containing all said insertion points. It may be compared to the region which would be delimited by a rubber band exclusively resting on these insertion points. A convex surface E is represented, for instance, in FIG. 3.

The “main axis” of a surface is the direction perpendicular to this surface passing through its geometrical centre (i.e. the barycentre, while considering that all the points have the same weight). The front view of a cup-shaped contact surface is a view, from the inside, along the main axis of this contact surface.

“cup-shaped” means concave as viewed from the center of the eye.

In the present description, unless otherwise stated, “comprising a” should be understood as “comprising at least one”.

### DETAILED DESCRIPTION

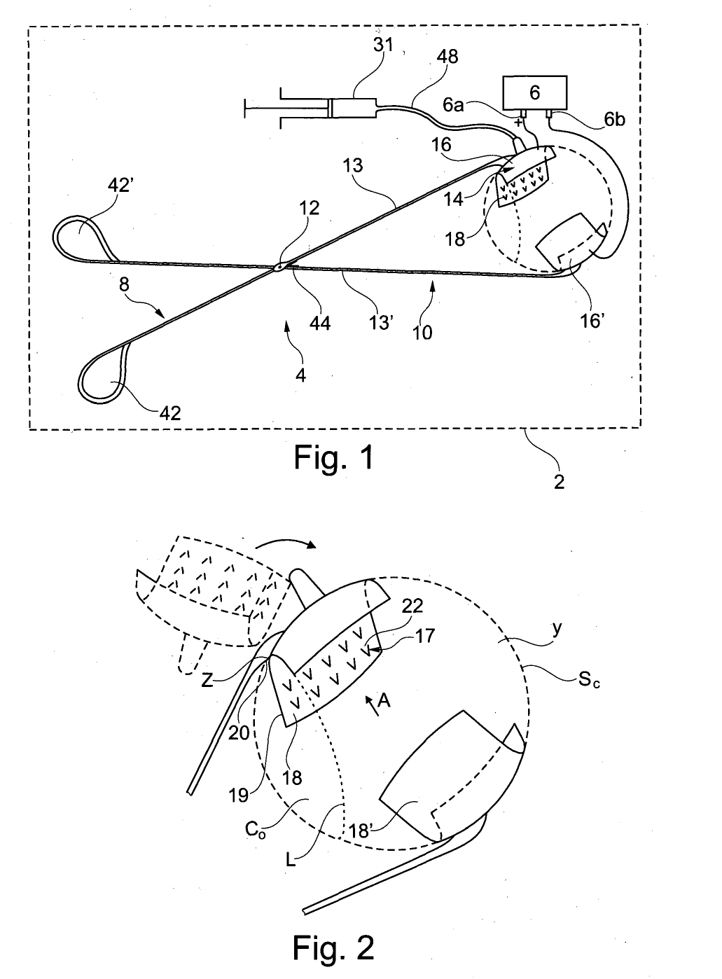

The electroporation device 2 shown in FIG. 1 comprises a scissor-like injection device 4 according to the invention, and an electrical generator 6.

As shown in FIGS. 2 and 3, the injection device 4 comprises a first arm 8 and a second arm 10, rotationally mounted on the first arm 8, around an axis 12. The first arm 8 and the second arm 10 are used as first and second electrodes of the electroporation device 2, respectively, the first arm being also used for the injection.

### First Arm

The first arm 8 comprises a first proximal part 13, and a first support 14, preferably made of a conductive material, presenting a first cup 16 provided with a set of injection needles 17.

The first support 14 is rotationally mounted on the first proximal part 13 around an axis Z. The axis Z is substantially perpendicular to the general direction of the injection needles 17.

The distance between any injection needle 17 and the axis Z is preferably more than 4 mm, preferably more than 5 mm.

### First Contact Surface

The first cup 16 defines a first contact surface 18, intended to come into contact with the outside surface of the eye, to limit the penetration depth of the injection needles which are described hereafter.

Because the first contact surface is designed to be placed in contact with the surface of an eye, it preferably has a smooth surface and, more preferably, a surface without any roughness.

The first cup may be rigid but it is preferably made of a flexible material, that is to say a material that is not aggressive with respect to the surface of the eye, for example polymers of silicone, of sponge, in particular synthetic sponge, of polyester, of polyorthoester, of polymethyl methacrylate or of any other flexible medical-grade polymers.

The maximal thickness of the first cup 16 is preferably less than 3 mm, less than 2 mm, less than 1 mm, or less than 0.5 mm.

Preferably, the flexibility of the first cup 16 is such that it may be deformed so that the first contact surface may match the outside surface of eyes having slightly different shapes or sizes.

The risk of leaking of the pharmaceutical composition during its injection in the suprachoroidal space is also limited.

In the represented embodiment, the first contact surface 18 extends along the substantially spherical envelope S, matching the outside surface of an eye, and preferably the slightly ellipsoidal outside surface of the anterior or posterior part of an eye. The first contact surface 18 has the form of a spheroidal or ellipsoidal band.

The first contact surface 18 may have two large sides 18₁ and 18₂ and two small sides 18₃ and 18₄. The large sides can in particular form rounded corners with the small sides.

The length of the small sides and/of the large sides may be greater than or equal to 5 mm, greater than or equal to 6 mm, and/or less than 20 mm, less than 18 mm, less than 15 mm, or less than 12 mm.

Seen from the front, as represented for example on FIG. 3, the first contact surface is externally defined by an outer contour O. The outer contour O is substantially rectangular in the preferred represented embodiment, but it is not limited to a rectangular contour.

The first contact surface may be solid or may be locally perforated (e.g. by holes). Preferably, the first contact surface is continuous, i.e. is not perforated.

The first contact surface 18 is, at least partially, preferably completely, defined by an electrically conductive material. Therefore, the first contact surface 18 may be part of a first electrode, or constitute a first electrode.

In particular, the first contact surface may be defined, at least partially, preferably completely, by a coating made of an electrically conductive material. The first support may also be made of an electrically conductive material, at least partially, so as to define the first contact surface.

The first contact surface may also comprise a plurality of pins connected electrically to one another and spread, preferably regularly, on the first contact surface 18.

### Locating Mark

The device preferably comprises a locating mark 19. The locating mark may advantageously match the surface of the eye.

The locating mark allows the operator to position the injection device, with remarkable precision, on the surface of the eye before any penetration of the injection needles through the outside surface thereof. The risk of error is therefore reduced or substantially eliminated.

In one embodiment, the locating mark is designed in such a way as to remain in contact with the outside surface of the eye during the penetration of the injection needles so as to guide this penetration. In particular, the locating mark may serve as a point of rotation for the injection needles during said penetration.

The locating mark may have different shapes. In particular, the locating mark may be formed by a point, at least 2 points, at least 3 points, or by all or part of a line, called “locating line”, or by a surface.

Preferably, the locating mark follows an arc of a circle so that the operator may position it in contact with the outside surface of the eye, substantially parallel to the edge of the cornea Co, that is to say of the transition shoulder between the cornea and the sclera Sc, called the “limbus” L.

The locating mark is preferably configured to be be placed on the outside surface of the eye, in contact with the limbus before the penetration of the injection needles into the eye. Advantgeously, it may be kept in contact with said limbus during the penetration of the injection needles through the outside surface of the eye, guiding this penetration.

The arc of a circle of the locating mark may have a radius of greater than 5 mm, greater than 6 mm, and/or of less than 8 mm or less than 7 mm, a radius of 6.58 mm being preferred.

The arc of a circle of the locating mark may extend, around its axis, on more than 80°, more than 100°, more than 120°, more than 140°, more than 160°. In an embodiment, it may extend on more than 180°, more than 200°, more than 220°, more than 240°, more than 260°, more than 280°, more than 300°.

The length of the locating line that is in contact with the outside surface of the eye may depend on the degree of penetration of the injection needles. For example, at the start of the stage of penetration, the locating line may be in contact via one or more points, or one or more fractions of this line, with the outside surface of the eye. During the penetration, the nature of this contact may evolve. Preferably, the length of the locating mark is sufficiently short to ensure that, given the flexibility of the eye, the locating mark may remain along its entire length in contact with the outside surface of the eye, throughout the stage of penetration.

The locating mark preferably has a smooth surface and, more preferably, a surface without any roughness, especially in the form of sharp tips or edges that could damage the surface of the eye during the stage of penetration.

The locating mark may in particular be formed by a band of flexible material with a width of greater than 1.5 mm and/or less than 5 mm extending, for example, along a side, for example along the entire length, of the first contact surface. The locating mark may in particular be formed by a bead of silicone or of foam. Advantageously, the risk of injury to the limbus (edge of the cornea) is thereby reduced.

The locating mark may be formed by or at least partially covered by a non-slip material that is able to limit the sliding movement on the surface of the eye.

Preferably, at least one side of the first contact surface, preferably all the sides of the first contact surface are delimited by an intersection of a plane with the spherical envelope S, as shown in FIG. 4, or with an ellipsoidal envelope. Preferably the locating line is defined by such a side of the first contact surface. It may also be defined by other parts of the injection device.

The axis Z of the rotation of the first support 14 on the first proximal part 13 preferably extends parallel to the plane of the locating mark 19. The distance between the axis Z and this plane is preferably less than 3 mm, less than 2 mm, less than 1 mm, or less than 0.5 mm.

Preferably, the first support 14 is mounted on the first proximal part 13 so that the first support may rotate between a disengaged position where the injection needles are not penetrating in the eye (represented with a dashed line) and an engaged position where the injection needles are introduced in the eye, while allowing the locating mark remaining in contact with the limbus of the eye during the rotation between these two positions.

The injection device preferably comprises desactivable means to lock the first support in the engaged position and/or in the disengaged position. The injection device may comprise elastic means, for instance a spring 20, acting so as to push the first support toward the engaged position. Such elastic means advantageously improve the contact of the first support with the outside surface of the eye, in particular during the injection and the electroporation.

Preferably, the spring 20 is mounted around the axis Z.

### Injection Needles

A set of parallel identical and rectilinear injection needles 17, extending along a common general direction W, are fixed on the first contact surface, for example by clipping, by adhesive bonding or by fusion of material. The injection needles may all be fixed in the same way on the first support, or not.

The external diameter of an injection needle may be between 0.2 and 0.4 mm.

The external diameter of any injection needle is, for example, about 0.3 mm.

Each injection needle 17 extends from a proximal end 21, embedded in the support at a respective location called “insertion point” 22, to a free distal end 26. The distal end 26 has a bevelled tip for facilitating the penetration of the injection needle into the eye, and opens out via one or several axial and/or radial ejection orifices. In an embodiment, any injection needle is tapered, that is to say conical along its axis, and opens out axially.

Preferably, the ejection orifice of an injection needle, preferably of any injection needle, has a smaller diameter than the inside diameter of the injection needle. Preferably, the injection needle opens out laterally.

The distal ends 26 of all the injection needles extend substantially on a spherical envelope S1, as is shown in FIG. 4, concentrically with the spherical envelope S. The difference between the radius of the spherical envelopes S and S1, corresponding to the length of the injection needles, is determined so that, when the first contact surface 18 is in contact with the outside surface of an eye, thus preventing further penetration of the injection needles into the eye, the ejection orifices of the injection needles are within the suprachoroidal space.

The injection needles may protrude from the first contact surface by a distance greater than 0.8 mm, and/or less than 1.2 mm, or less than 1.1 mm, or less than 1.0 mm.

They all extend parallel to the main axis of the first contact surface.

The may also extend perpendicular to the first contact surface. All the injection needles may have the same length and be oriented toward the center of the spherical envelope S.

The insertion points 22 of the injection needles 17 on the first contact surface 18 are spread on this surface. They are not all aligned.

In an embodiment, the insertion points are spread on the first contact surface 18 along several straight lines, in particular along several rows and columns, which may be perpendicular to each other or not. For example, in FIG. 3, the insertion points are spread along three rows and six columns.

The distribution of the insertion points 22 may be homogeneous or not. However, there should not be any large area not containing any insertion point within the convex surface E defined by the insertion points 22, i.e. in the region where the insertion points are spread, and preferably within the whole first contact surface 18.

Preferably, the injection needles are configured so that, when the locating mark is positioned bearing on the limbus, no injection needle may penetrate into the outside surface of the eye at a distance “d” less than 4 mm, preferably less than 5 mm from the limbus. In particular, all the insertion points of the injection needles are preferably more than 4 mm, preferably more than 5 mm away from the locating mark.

Preferably, the distance between the locating mark and the closest point of penetration of an injection needle into the eye, and/or between the locating mark and the closest insertion point of an injection needle is less than 10 mm, less than 8 mm or less than 6 mm. The efficiency of the first support is advantageously increased.

FIG. 3 shows a front view of the first contact surface 18, i.e. as observed according to arrow A. The convex surface E and the outer contour O of the first contact surface 18 are represented.

The largest dimension and/or the smallest dimension of the convex surface E, in said front view, is (are) preferably more than 9 mm, more than 10 mm, and/or less than 30 mm, less than 20 mm, less than 18 mm or less than 15 mm.

The diameter D of the largest circle C that it is possible to place, in the surface defined by the outer contour O, on a front view, without any insertion point being included in said circle, should be less than 12 mm.

Preferably, the diameter D′ of the largest circle C′ that it is possible to place, in the convex surface E, on a front view, without any insertion point being included in said circle should be less than 12 mm.

In combination with the shape and dimensions of the first contact surface and of the injection needles, a distribution of the insertion points according to the invention enables a precise and homogeneous injection within the suprachoroidal space, without having to place the pharmaceutical composition under high pressure. The injury of the eye is therefore reduced and the electroporation is very efficient.

At least some, preferably all the injection needles may be made at least in part, or entirely, of an electrically conductive material, and connected together so as to belong to one and the same electrode, i.e. the first electrode.

According to some embodiments, all the injection needles all electrically connected together, possibly with the first contact surface, so as to constitute one single electrode.

According to some embodiments, the first electrode only comprises the first contact surface, the injection needles being electrically isolated from the first contact surface.

The first arm 8 also comprises a first connector for connecting the first electrode, i.e. the injection needles and/or the first contact surface, to a first terminal 6a of the electrical generator 6.

Preferably, at least one injection needle, preferably all the injection needles are provided with a respective optical fibre so that the operator may visually evaluate their depth of penetration in the eye. The light provided by the optical fibre(s) is preferably a cold light, i.e. providing substantially no heat.

### Fluid Communication

Each injection needle 17 is traversed, in the normal way, by a lumen which is designed for the transfer of a pharmaceutical composition (examples of which are given hereafter) from the proximal end to the distal end of the injection needle.

The lumens of the injection needles open into a common distribution chamber 30.

The distribution chamber 30 is preferably formed in the first support.

The electroporation device comprises a reservoir of the pharmaceutical composition that is to be injected, for instance a reservoir integral with the first support, or, as shown in FIG. 1, a reservoir in the form of a syringe 31. The syringe 31 may be in fluid communication, through a tube 48 for instance plugged on a Luer cone 32 of the first support, with the distribution chamber 30 in fluid communication with all the injection needles. An action on the piston of the syringe transfers the pharmaceutical composition out of the reservoir into the distribution chamber. The distribution chamber 30 allows the pharmaceutical composition to be simultaneously delivered to all injection needles.

According to some embodiments, the injection device has means for selectively or simultaneously plugging one or more, preferably all, of the lumens of the injection needles. These means may in particular comprise one or more stoppers, each designed to plug one or more injection needles.

According to some embodiments, each injection needle may be supplied independently of the others. For example, each injection needle may be connected to an individual tube. It is thus advantageously possible to inject different active principles through the different injection needles of the device.

In this embodiment, it is possible to stop the supply to the associated injection needle by clamping or pinching a tube.

All the tubes may also be connected to a main tube which, when clamped or pinched, causes the supply to all the tubes to be cut simultaneously.

### Proximal Part

The first proximal part 13 is intended for manipulation of the injection device, allowing the injection device to be gripped, for example, between a thumb and an index finger of one hand. Manipulation of the injection device is made easier in this way.

The first proximal part 13 preferably presents a first orifice 42 enabling the introduction of a finger, facilitating the manipulation of the first arm.

The first support 14 is mounted at the end of the first proximal part which is opposite to the orifice 42.

The first proximal part 13 is preferably made of or covered with a non-conductive material.

### Second Arm

The second arm 10 is similar to the first arm 8. In the various figures, identical reference signs are used to designate identical or similar elements of the first arm and the second arm. However, the reference signs are complemented by a ′ sign for the second arm.

The distal part of the second arm 10 comprises a second support 14′, preferably made of or coated with an electrically conductive material, presenting a second cup 16′. Preferably, the second support has one or more of the characteristics of the first support.

The second cup 16′ defines a preferably substantially spherical second contact surface 18′, matching the outside surface of an eye, and preferably a slightly ellipsoidal second contact surface, matching the anterior or posterior part of the outside surface of an eye.

Preferably, the second support 14′ presents an electrically conductive second contact surface 18′ similar to the first electrically conductive contact surface 18, preferably disposed on the second support in a similar way as the second electrically conductive first contact surface on the first support.

The second contact surface 18′ is intended to be used as the second electrode for the electroporation device 2.

The second electrode, and even the second arm 8, preferably do not comprise any injection needle. The second electrode is preferably a surface electrode, i.e. designed so as to not penetrate into the eye.

The second arm 10 comprises a second connector for connecting the second contact surface 18′, defined by an electrically conductive material, to a second terminal 6b of the electrical generator 6.

The injection device is preferably configured so that the first and second electrically conductive contact surfaces are concentric in a position corresponding to the close position where they bear on opposite regions of the outside surface of an eye Y, relative to the centre of the eye, as shown in FIG. 1.

The proximal part of the second arm 8 comprises a second proximal part 13′, preferably made of a non-conductive material, presenting a second orifice 42′, similar to the first orifice 42, facilitating the manipulation of the second arm. The second support 14′ is rigidly fixed at the end of the proximal part of the second arm which is opposite to the orifice 42′.

The rotational movement of the second arm relative to the first arm around the axis 12 may lead the second arm in a close position, where the first and second contact surfaces may “pinch” or “clamp” the eye Y, two opposite regions of the outside surface of the eye Y being in close contact with the first and second contact surfaces, respectively, preferably acting as first and second electrodes, respectively.

A spring 44 preferably tends to push the first arm toward the second arm. Preferably, this spring is mounted around the axis 12.

The second arm may also be provided with an optical fibre and/or a plurality of electroluminescent diods (LED) may be disposed on the second electrod, so that the operator may evaluate the depth of penetration of the injection needles through a transillumination. The light provided by this optical fibre is preferably a cold light.

### Pharmaceutical Composition

An electroporation device according to the invention may be used for the electroporation of a therapeutic nucleic acid of interest after delivering a pharmaceutical composition formulated with said therapeutic nucleic acid into the suprachoroidal space of a diseased eye.

The nucleic acid to be used in the instant invention can be any nucleic acid of interest exhibiting a biological property. More particularly, the nucleic acid can be any nucleic acid encoding a natural, truncated, artificial, chimeric or recombinant product [e.g., a polypeptide of interest (including a protein or a peptide), a RNA, etc.] exhibiting a biological activity.

The nucleic acid is preferably a desoxyribonucleic acid (DNA) molecule (cDNA, gDNA, synthetic DNA, artificial DNA, recombinant DNA, etc.) or a ribonucleic acid (RNA) molecule (mRNA, tRNA, RNAi, RNAsi, catalytic RNA, antisens RNA, viral RNA, etc.). The nucleic acid may be single stranded or multistranded nucleic acid, preferably double-stranded nucleic acid or may be complexed. The nucleic acid may comprise hybrid sequences or synthetic or semi-synthetic sequences. It may be obtained by any technique known to persons skilled in the art, and especially by screening libraries, by chemical synthesis, or alternatively by mixed methods including chemical or enzymatic modification of sequences obtained by screening libraries.

In a particular embodiment, the therapeutic nucleic acid is of synthetic or biosynthetic origin, or extracted from a virus or from a unicellular or pericellular eukaryotic or prokaryotic organism.

The therapeutic nucleic acid used in the present invention may be naked, may be complexed with any chemical, biochemical or biological agent, may be inserted in a vector, etc., when administered to the suprachoroidal space.

As used herein, the term “naked DNA” refers to any nucleic acid molecule which is not combined with a synthetic, biosynthetic, chemical, biochemical or biological agent improving the delivery or transfer of said DNA, or facilitating its entry into the cell.

As used herein, the term “vector” refers to a nucleic acid molecule capable of transporting another nucleic acid to which it has been linked. This term also refers in the present application to any delivery carrier, such as a composition associated to a therapeutic or prophylactic nucleic acid in order to increase its cellular delivery.

Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer to circular double stranded DNA loops which, in their vector form, are not bound to the chromosome. In the present invention, the plasmid is the most commonly used form of vector. The plasmid is a preferred form of naked DNA according to the invention.

Vectors may also be episomal DNA, yeast artificial chromosomes, minichromosomes or viral vectors wherein the viral vector is selected from the group consisting of a lentivirus, an adenovirus, an adeno-associated virus and a virus-like vector.

The vector may also be a lipid vesicle such as a liposome. Lipid based compounds which are not liposomes may further be used. For example, lipofectins and cytofectins are lipid-based positive ions that bind to negatively charged nucleic acid and form a complex that can ferry the DNA across a cell membrane. The invention is intended to include such other forms of expression vectors which serve equivalent functions and which become known in the art subsequently hereto.

In addition, the nucleic acid according to the invention may also contain one or more additional regions, for example regulatory elements of small or large size which are available to the skilled artisan such as a promoter region (constitutive, regulated, inducible, tissue-specific, etc.), for example sequences allowing and/or promoting expression in the targeted tissue (e.g. choroid or retina) or cells (e.g. RPE or photoreceptors), a transcription termination signal, secretion sequences, an origin of replication and/or nuclear localization signal (nls) sequences which further enhance polynucleotide transfer to the cell nucleus. Such nls sequences have been described in the prior art including the SV40 large T antigen sequence.

Additionally, the nucleic acid may further comprise selectable markers useful in selecting, measuring, and monitoring nucleic acid transfer results (transfer to which tissues, duration of expression, etc.). The types of expression systems and reporter genes that can be used or adapted for use are well known in the art. For example, genes coding for a luciferase activity, an alkaline phosphatase activity, or a green fluorescent protein activity are commonly used.

The nucleic acid according to the invention may contain any nucleotide sequence of any size. The nucleic acid may thus vary in size from a simple oligonucleotide to a larger molecule such as a nucleotide sequence including exons and/or introns and/or regulatory elements of any sizes (small or large), a gene of any size, for example of large size, or a chromosome for instance, and may be a plasmid, an episome, a viral genome, a phage, a yeast artificial chromosome, a minichromosome, an antisense molecule, etc.

In a particularly preferred embodiment, the polynucleotide is a double-stranded, circular DNA, such as a plasmid, encoding a product with biological activity.

The nucleic acid can be prepared and produced according to conventional recombinant DNA techniques, such as amplification, culture in prokaryotic or eukaryotic host cells, purification, etc. The techniques of recombinant DNA technology are known to those of ordinary skill in the art.

In a particular embodiment, the nucleic acid of interest is capable of exerting a beneficial effect on the targeted cells. It may compensate for a deficiency in or reduce an excess of an endogenous substance. Alternatively, it may confer new properties on the targeted cells. It may be for example an antisense sequence or nucleic acid encoding a polypeptide which can affect the function, morphology, activity and/or metabolism of ocular cells.

The down regulation of gene expression using antisense nucleic acids can be achieved at the translational or transcriptional level. Antisense nucleic acids of the invention are preferably nucleic acid fragments capable of specifically hybridizing with a nucleic acid encoding an endogenous ocular active substance or the corresponding messenger RNA. These antisense nucleic acids can be synthetic oligonucleotides, optionally modified to improve their stability and selectivity. They can also be DNA sequences whose expression in the cell produces RNA complementary to all or part of the mRNA encoding an endogenous ocular active substance. Antisense nucleic acids can be prepared by expression of all or part of a nucleic acid encoding an endogenous ocular active substance, in the opposite orientation. Any length of antisense sequence is suitable for practice of the invention so long as it is capable of down-regulating or blocking expression of the endogenous ocular active substance. Preferably, the antisense sequence is at least nucleotides in length. The preparation and use of antisense nucleic acids, DNA encoding antisense RNAs and the use of oligo and genetic antisense is disclosed in WO92/15680, the content of which is incorporated herein by reference.

Among the biologically active polypeptides or proteins optionally expressed by a nucleic acid as described above and suitable for practice of the invention are enzymes, blood derivatives, hormones, lymphokines, cytokines, chimiokines, anti-inflammatory factors, growth factors, trophic factors, neurotrophic factors, haematopoietic factors, angiogenic factors, anti-angiogenic factors, inhibitors of metalloproteinase, regulators of apoptosis, coagulation factors, receptors thereof, in particular soluble receptors, a peptide which is an agonist or antagonist of a receptor or of an adhesion protein, antigens, antibodies, fragments or derivatives thereof and other essential constituents of the cell, proteins involved in the visual cycle within RPE cells, and structure proteins of retinal cells.

Various retina-derived neurotrophic factors have the potential to rescue degenerating photoreceptor cells, and may be delivered trough a method according to the present invention. Preferred biologically active agents may be selected from VEGF, Angiogenin, Angiopoietin-1, DeM, acidic or basic Fibroblast Growth Factors (aFGF and bFGF), FGF-2, Follistatin, Granulocyte Colony-Stimulating factor (G-CSF), Hepatocyte Growth Factor (HGF), Scatter Factor (SF), Leptin, Midkine, Placental Growth Factor (PGF), Platelet-Derived Endothelial Cell Growth Factor (PD-ECGF), Platelet-Derived Growth Factor-BB (PDGF-BB), Pleiotrophin (PTN), RdCVF (Rod-derived Cone Viability Factor), Progranulin, Proliferin, Transforming Growth Factor-alpha (TGF-alpha), Transforming Growth Factor-beta (TGF-beta), Tumor Necrosis Factor-alpha (TNF-alpha), Vascular Endothelial Growth Factor (VEGF), Vascular Permeability Factor (VPF), CNTF, BDNF, GDNF, PEDF, NT3, BFGF, angiopoietin, ephrin, EPO, NGF, IGF, GMF, aFGF, NT5, Gax, a growth hormone, [alpha]-1-antitrypsin, calcitonin, leptin, an apolipoprotein, an enzyme for the biosynthesis of vitamins, hormones or neuromediators, chemokines, cytokines such as IL-1, IL-8, IL-10, IL-12, IL-13, a receptor thereof, an antibody blocking any one of said receptors, TIMP such as TIMP-1, TIMP-2, TIMP-3, TIMP-4, angioarrestin, endostatin such as endostatin XVIII and endostatin XV, ATF, angiostatin, a fusion protein of endostatin and angiostatin, the C-terminal hemopexin domain of matrix metalloproteinase-2, the kringle 5 domain of human plasminogen, a fusion protein of endostatin and the kringle 5 domain of human plasminogen, the placental ribonuclease inhibitor, the plasminogen activator inhibitor, the Platelet Factor-4 (PF4), a prolactin fragment, the Proliferin-Related Protein (PRP), the antiangiogenic antithrombin III, the Cartilage-Derived Inhibitor (CDI), a CD59 complement fragment, vasculostatin, vasostatin (calreticulin fragment), thrombospondin, fibronectin, in particular fibronectin fragment gro-beta, an heparinase, human chorionic gonadotropin (hCG), interferon alpha/beta/gamma, interferon inducible protein (IP-10), the monokine-induced by interferon-gamma (Mig), the interferon-alpha inducible protein 10 (IP10), a fusion protein of Mig and IP10, soluble Fms-Like Tyrosine kinase 1 (FLT-1) receptor, Kinase insert Domain Receptor (KDR), regulators of apoptosis such as Bcl-2, Bad, Bak, Bax, Bik, BcI-X short isoform and Gax, fragments or derivatives thereof and the like.

In a particular embodiment, the nucleic acid encodes a soluble fragment of the TNF[alpha] receptor, the TGF[beta]2 receptor, of VEGFR-1, VEGFR-2, VEGFR-3, CCR2 or MIP1. The nucleic acid may also, in another preferred embodiment, encode an antibody, a variable fragment of a single-chain antibody (ScFv) or any other antibody fragment having recognition capacities for the purposes of immunotherapy.

In a particular embodiment of the present invention, the biologically active nucleic acid encodes a precursor of a therapeutic protein usable in the present invention such as those described above.

In another particular embodiment, the electroporation device of the invention is particularly suitable for performing gene replacement. Accordingly the nucleic acid may encode for a viable protein so as to replace the defective protein which is naturally expressed in the targeted tissue. Typically, defective genes that may be replaced include, but are not limited to, genes that are responsible for retinal degenerative diseases such as retinitis pigmentosa (RP), Leber congenital amaurosis (LCA), recessive RP, Dominant retinitis pigmentosa, X-linked retinitis pigmentosa, Incomplete X-linked retinitis pigmentosa, dominant, Dominant Leber congenital amaurosis, Recessive ataxia, posterior column with retinitis pigmentosa, Recessive retinitis pigmentosa with para-arteriolar preservation of the RPE, Retinitis pigmentosa RP12, Usher syndrome, Dominant retinitis pigmentosa with sensorineural deafness, Recessive retinitis punctata albescens, Recessive Alström syndrome, Recessive Bardet-Biedl syndrome, Dominant spinocerebellar ataxia w/macular dystrophy or retinal degeneration, Recessive abetalipoproteinemia, Recessive retinitis pigmentosa with macular degeneration, Recessive Refsum disease, adult form, Recessive Refsum disease, infantile form, Recessive enhanced S-cone syndrome, Retinitis pigmentosa with mental retardation, Retinitis pigmentosa with myopathy, Recessive Newfoundland rod-cone dystrophy, Retinitis pigmentosa sinpigmento, Sector retinitis pigmentosa, Regional retinitis pigmentosa, Senior-Loken syndrome, Joubert syndrome, Stargardt disease, juvenile, Stargardt disease, late onset, Dominant macular dystrophy, Stargardt type, Dominant Stargardt-like macular dystrophy, Recessive macular dystrophy, Recessive fundus flavimaculatus, Recessive cone-rod dystrophy, X-linked progressive cone-rod dystrophy, Dominant cone-rod dystrophy, Cone-rod dystrophy; de Grouchy syndrome, Dominant cone dystrophy, X-linked cone dystrophy, Recessive cone dystrophy, Recessive cone dystrophy with supernormal rod electroretinogram, X-linked atrophic macular dystrophy, X-linked retinoschisis, Dominant macular dystrophy, Dominant radial, macular drusen, Dominant macular dystrophy, bull's-eye, Dominant macular dystrophy, butterfly-shaped, Dominant adult vitelliform macular dystrophy, Dominant macular dystrophy, North Carolina type, Dominant retinal-cone dystrophy 1, Dominant macular dystrophy, cystoid, Dominant macular dystrophy, atypical vitelliform, Foveomacular atrophy, Dominant macular dystrophy, Best type, Dominant macular dystrophy, North Carolina-like with progressive, Recessive macular dystrophy, juvenile with hypotrichosis, Recessive foveal hypoplasia and anterior segment dysgenesis, Recessive delayed cone adaptation, Macular dystrophy in blue cone monochromacy, Macular pattern dystrophy with type II diabetes and deafness, Flecked Retina of Kandori, Pattern Dystrophy, Dominant Stickler syndrome, Dominant Marshall syndrome, Dominant vitreoretinal degeneration, Dominant familial exudative vitreoretinopathy, Dominant vitreoretinochoroidopathy; Dominant neovascular inflammatory vitreoretinopathy, Goldmann-Favre syndrome, Recessive achromatopsia, Dominant tritanopia, Recessive rod monochromacy, Congenital red-green deficiency, Deuteranopia, Protanopia, Deuteranomaly, Protanomaly, Recessive Oguchi disease, Dominant macular dystrophy, late onset, Recessive gyrate atrophy, Dominant atrophia greata, Dominant central areolar choroidal dystrophy, X-linked choroideremia, Choroidal atrophy, Central areolar, Central, Peripapillary, Dominant progressive bifocal chorioretinal atrophy, Progresive bifocal Choroioretinal atrophy, Dominant Doyne honeycomb retinal degeneration (Malattia Leventinese), Amelogenesis imperfecta, Recessive Bietti crystalline corneoretinal dystrophy, Dominant hereditary vascular retinopathy with Raynaud phenomenon and migraine, Dominant Wagner disease and erosive vitreoretinopathy, Recessive microphthalmos and retinal disease syndrome; Recessive nanophthalmos, Recessive retardation, spasticity and retinal degeneration, Recessive Bothnia dystrophy, Recessive pseudoxanthoma elasticum, Dominant pseudoxanthoma elasticum; Recessive Batten disease (ceroid-lipofuscinosis), juvenile, Dominant Alagille syndrome, McKusick-Kaufman syndrome, hypoprebetalipoproteinemia, acanthocytosis, palladial degeneration; Recessive Hallervorden-Spatz syndrome; Dominant Sorsby's fundus dystrophy, Oregon eye disease, Kearns-Sayre syndrome, Retinitis pigmentosa with developmental and neurological abnormalities, Basseb Korenzweig Syndrome, Hurler disease, Sanfilippo disease, Scieie disease, Melanoma associated retinopathy, Sheen retinal dystrophy, Duchenne macular dystrophy, Becker macular dystrophy, and Birdshot Retinochoroidopathy. Examples of genes include but are not limited to genes encoding for ATP-binding cassette transporter, RPE65, RdCVF, CP290 . . . .

In another embodiment, the electroporation device of the invention is particularly suitable for performing exon skipping for restoring the function of mutated proteins responsible for retinal degenerative disease. Exon skipping involves blocking or preventing the incorporation into mature mRNA of one or more targeted exon(s) which encodes amino sequences that are responsible for a protein dysfunction. This is accomplished by exposing the pre-mRNA that includes exons encoding the protein to antisense oligonucleotides (AONs) which are complementary to sequence motifs that are required for correct splicing of the one or more targeted exons. The AONs bind to complementary required sequences in the pre-mRNA and prevent normal splicing. Instead, the targeted exons are excised and are not included in the mature mRNA that is translated into protein, and the amino acid sequences encoded by the targeted exons are missing from the translated protein.

Furthermore, in another embodiment of the present invention, a mixture of nucleic acids encoding distinct biologically active products can be used. This variant allows co-expression of different products in the ocular cells.

The pharmaceutical composition of the invention may also comprise compatible or physiologically acceptable carrier, excipient or diluent.

The term “pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

Pharmaceutically compatible or physiologically acceptable carrier, excipient or diluent includes diluents and fillers which are pharmaceutically acceptable for the methods of the invention, are sterile, and may be selected from neutral to slightly acidic, isotonic, buffered saline (including phosphates, chloride, etc.), aqueous or oleaginous solutions or suspensions and more preferably from sucrose, trehalose, surfactants, proteins and amino acids. The pharmaceutically compatible or physiologically acceptable carrier, excipient or diluent is preferably formulated using suitable dispersing, wetting, suspending, soothing, isotonic or viscosity building agents, stabilizers, preservatives and appropriate buffers to form an isotonic solution. The particular pharmaceutically acceptable carrier and the ratio of active compound to carrier are determined by the solubility and chemical properties of the composition, the particular mode of administration, and standard pharmaceutical practice. Those skilled in the art will understand how to formulate such vehicles by known techniques.

An example of stabilizers is disodium edetate or the like. Examples of isotonic agents are glycerin, propylene glycol, polyethylene glycol, sodium chloride, potassium chloride, sorbitol and mannitol or the like. Examples of buffers are citric acid, sodium hydrogenphosphate, glacial acetic acid and trometamol or the like. Examples of pH adjusters are hydrochloric acid, citric acid, phosphoric acid, acetic acid, sodium hydroxide, sodium carbonate and sodium hydrogencarbonate or the like. An example of soothing agents is benzyl alcohol or the like. Examples of preservatives are benzalkonium chloride, benzethonium chloride, p-hydroxybenzoate esters, sodium benzoate and chlorobutanol or the like.

Viscosity greater than that of simple aqueous solutions may be desirable to increase ocular absorption of the active compound, to decrease variability in dispensing the formulations, to decrease physical separation of components of a suspension or emulsion of formulation and/or otherwise to improve the ophthalmic formulation. Such viscosity building agents include, for example, polyvinyl alcohol, polyvinyl pyrrolidone, methyl cellulose, hydroxypropyl methylcellulose, hydroxyethyl cellulose, carboxymethyl cellulose, hydroxypropyl cellulose or other agents known to those skilled in the art. Such agents are typically employed at a level of from about 0.01 to about 2 wt. %.

Preparation forms of the pharmaceutical composition intended for administration to suprachoroidal space are preferably liquid preparations. The liquid preparations can be prepared, for example, by dissolving the biologically active agent in BSS (Balanced Salt Solution), a glycerin solution, a hyaluronic acid solution and the like. A particular composition comprises for example BBS (60%) and hyaluronic acid (40%). A stabilizer, an isotonic agent, a buffer, a pH adjustor, a soothing agent, a preservative, an injectable viscuous polymer, such as a polyorthoester or a polyanhydride, electrolytes, such as sodium, potassium, calcium, magnesium and/or chloride or the like may optionally be added in an adequate amount to the liquid preparations.

The pharmaceutical composition may comprise or the biologically active agent may be combined (in a use according to the present invention) with any additional active ingredient or adjuvant. The adjuvant may be selected from any substance, mixture, solute or composition facilitating or increasing the biological activity of the prophylactic or therapeutic agent such as any biologic, synthetic or biosynthetic agent which improves the delivery or transfer of said agent and may be assimilated to a vector (as delivery carrier) according to the invention. The adjuvant may be conditioned and administered separately or sequentially from the prophylactic or therapeutic agent containing composition and/or at a distinct site of injection. Treatment with multiple agents and/or adjuvants according to the invention need not be done using a mixture of agents and/or adjuvants but may be done using separate pharmaceutical preparations. The preparations need not be delivered at the same exact time, but may be coordinated to be delivered to a patient during the same period of treatment, i. e., within a week or a month of each other.

Any suitable therapeutic agents can be coordinated with the compositions of the present invention. Non-limiting examples of therapeutic agents which may be administered in addition to the above biologically active (prophylactic or therapeutic) agent(s) through a method according to the present invention also include permeabilizing agents such as a virus, a lipid vesicle, hyaluronic acid, lipid-based positive ions, polycationic emulsions, cationic peptides, polyplex, etc.; Actual dosage levels of active ingredients in the compositions of the present invention may be adapted so as to obtain an amount of active ingredient that is effective to obtain a desired biological activity. It should be understood, however, that the specific dose level for any particular patient will depend upon a variety of factors including the body weight, general health, sex, diet, time, rates of absorption and excretion, combination with other drugs and the severity of the particular disease being treated.

### Kit

In accordance with the present invention, kits for preventing or treating an ocular disease are envisioned. An injection device according to the invention and a pharmaceutical composition according to the invention, and optionally a counter electrode, optionally an electrical generator, optionally instructions for use may be supplied together in a kit. Within the kit, the components may be separately packaged or contained.

Instructions can be in written, video, or audio form, and can be contained on paper, an electronic medium, or even as a reference to another source, such as a website or reference manual.

Other components such as excipients, carriers, other drugs or adjuvants, instructions for administration of the active substance or composition, and administration or injection devices can be supplied in the kit as well.

The kit may also contain means to light the injection device in operation, in particular an optical fibre. The light provided by this optical fibre is preferably a cold light.

### Method

The method of the invention may be used for treating an ocular disease in a subject, the pharmaceutical composition being preferably chosen among the pharmaceutical compositions which are described here above.

The method of the present invention is particularly suitable for the treatment of ocular diseases affecting the posterior region of the eye, and more particularly ocular diseases affecting the retina, and more specifically the macula. Non-limiting examples of ocular diseases that may be treated by the method of the present invention include ocular diseases affecting the macula such as age related macular degeneration (wet and dry) or inherited macular degeneration, macular oedema of any origin (age related macular degeneration, diabetes, inflammation, degeneration, central serous chorioretinitis or diffuse epitheliopathy, diabetic retinopathy . . . ), inherited retinal dystrophies, such as Leber congenital amaurosis, retinitis pigmentosa, cone rod dystophies, cone dystrophies, best vitelliforme maculopathy, intraocular inflammation such retinitis, chorioretinitis, choroiditis, ischemic retinopathy (in particular retinopathy of prematurity and diabetic retinopathy), retinal vascular diseases, ocular ischemia syndrome and other vascular anomalies, choroidal disorders and tumors, vitreous disorders, glial proliferation such as proliferative vitreo retinopathy and glial proliferation associated to diabetic pre retinal angiogenesis, diabetic retinopathy ischemic or proliferative.

Inherited retinal dystrophies or retinitis pigmentosa are inherited blinding diseases due to mutations or deletions in genes implicated in the visual cycle. They begin at a young age and progress slowly until total blindness. Loss of photoreceptors is associated with loss of retinal pigment cells and to vascular and optic nerve atrophy at the later stages. Some of these inherited degeneration are due to mutation in mitochondrial DNA. In particular, non limiting examples of retinal degenerative diseases include but are not limited to retinitis pigmentosa (RP), Leber congenital amaurosis (LCA), recessive RP, Dominant retinitis pigmentosa, X-linked retinitis pigmentosa, Incomplete X-linked retinitis pigmentosa, dominant, Dominant Leber congenital amaurosis, Recessive ataxia, posterior column with retinitis pigmentosa, Recessive retinitis pigmentosa with para-arteriolar preservation of the RPE, Retinitis pigmentosa RP12, Usher syndrome, Dominant retinitis pigmentosa with sensorineural deafness, Recessive retinitis punctata albescens, Recessive Alström syndrome, Recessive Bardet-Biedl syndrome, Dominant spinocerebellar ataxia w/ macular dystrophy or retinal degeneration, Recessive abetalipoproteinemia, Recessive retinitis pigmentosa with macular degeneration, Recessive Refsum disease, adult form, Recessive Refsum disease, infantile form, Recessive enhanced S-cone syndrome, Retinitis pigmentosa with mental retardation, Retinitis pigmentosa with myopathy, Recessive Newfoundland rod-cone dystrophy, Retinitis pigmentosa sinpigmento, Sector retinitis pigmentosa, Regional retinitis pigmentosa, Senior-Loken syndrome, Joubert syndrome, Stargardt disease, juvenile, Stargardt disease, late onset, Dominant macular dystrophy, Stargardt type, Dominant Stargardt-like macular dystrophy, Recessive macular dystrophy, Recessive fundus flavimaculatus, Recessive cone-rod dystrophy, X-linked progressive cone-rod dystrophy, Dominant cone-rod dystrophy, Cone-rod dystrophy; de Grouchy syndrome, Dominant cone dystrophy, X-linked cone dystrophy, Recessive cone dystrophy, Recessive cone dystrophy with supernormal rod electroretinogram, X-linked atrophic macular dystrophy, X-linked retinoschisis, Dominant macular dystrophy, Dominant radial, macular drusen, Dominant macular dystrophy, bull's-eye, Dominant macular dystrophy, butterfly-shaped, Dominant adult vitelliform macular dystrophy, Dominant macular dystrophy, North Carolina type, Dominant retinal-cone dystrophy 1, Dominant macular dystrophy, cystoid, Dominant macular dystrophy, atypical vitelliform, Foveomacular atrophy, Dominant macular dystrophy, Best type, Dominant macular dystrophy, North Carolina-like with progressive, Recessive macular dystrophy, juvenile with hypotrichosis, Recessive foveal hypoplasia and anterior segment dysgenesis, Recessive delayed cone adaptation, Macular dystrophy in blue cone monochromacy, Macular pattern dystrophy with type II diabetes and deafness, Flecked Retina of Kandori, Pattern Dystrophy, Dominant Stickler syndrome, Dominant Marshall syndrome, Dominant vitreoretinal degeneration, Dominant familial exudative vitreoretinopathy, Dominant vitreoretinochoroidopathy; Dominant neovascular inflammatory vitreoretinopathy, Goldmann-Favre syndrome, Recessive achromatopsia, Dominant tritanopia, Recessive rod monochromacy, Congenital red-green deficiency, Deuteranopia, Protanopia, Deuteranomaly, Protanomaly, Recessive Oguchi disease, Dominant macular dystrophy, late onset, Recessive gyrate atrophy, Dominant atrophia greata, Dominant central areolar choroidal dystrophy, X-linked choroideremia, Choroidal atrophy, Central areolar, Central, Peripapillary, Dominant progressive bifocal chorioretinal atrophy, Progresive bifocal Choroioretinal atrophy, Dominant Doyne honeycomb retinal degeneration (Malattia Leventinese), Amelogenesis imperfecta, Recessive Bietti crystalline corneoretinal dystrophy, Dominant hereditary vascular retinopathy with Raynaud phenomenon and migraine, Dominant Wagner disease and erosive vitreoretinopathy, Recessive microphthalmos and retinal disease syndrome; Recessive nanophthalmos, Recessive retardation, spasticity and retinal degeneration, Recessive Bothnia dystrophy, Recessive pseudoxanthoma elasticum, Dominant pseudoxanthoma elasticum; Recessive Batten disease (ceroid-lipofuscinosis), juvenile, Dominant Alagille syndrome, McKusick-Kaufman syndrome, hypoprebetalipoproteinemia, acanthocytosis, palladial degeneration; Recessive Hallervorden-Spatz syndrome; Dominant Sorsby's fundus dystrophy, Oregon eye disease, Kearns-Sayre syndrome, Retinitis pigmentosa with developmental and neurological abnormalities, Basseb Korenzweig Syndrome, Hurler disease, Sanfilippo disease, Scieie disease, Melanoma associated retinopathy, Sheen retinal dystrophy, Duchenne macular dystrophy, Becker macular dystrophy, and Birdshot Retinochoroidopathy.

Intraocular inflammation regroups all types of inflammation of the intraocular tissues, mainly uvea and retina. Intraocular inflammations may be from immunologic causes, infectious causes, iatrogenic causes or of unknown etiologies. They may be acute, recurrent or chronic. Intraocular inflammations are among the most common causes of curable blindness. Posterior segment intraocular inflammations may be associated with vasculitis, optic neuritis, vitritis and chorio retinitis, retinitis, choriditis, choroidal neovascularisation, choroidal neovascularization due to AMD, to myopia, inflammation, diffuse epitheliopathy, bruch membrane rupture, polypoidal choroidal vasculopathy, post traumatic . . . .

There are two major types of glaucoma: chronic glaucoma or primary open-angle glaucoma (POAG) and acute closed-angle glaucoma. Other variations include congenital glaucoma, pigmentary glaucoma, neovascular glaucoma and secondary glaucoma. Glaucoma is similar to ocular hypertension but with accompanying optic nerve damage and vision loss. Glaucoma is usually treated with eye drops, laser, or conventional eye surgery. If not treated, glaucoma will cause blindness.

Angiogenesis is the formation of new capillary blood vessels leading to neovascularization. Angiogenesis is a complex process which includes a series of sequential steps including endothelial cell mediated degradation of vascular basement membrane and interstitial matrices, migration of endothelial cells, proliferation of endothelial cells, and formation of capillary loops by endothelial cells. Though angiogenesis is a normal process for the development or maintenance of the vasculature, pathological conditions (i.e., angiogenesis dependent diseases) arise where blood vessel growth is actually harmful. Angiogenesis is notably associated with important diseases of ocular tissue, including diabetic retinopathies, age related macular degeneration, retinopathy of prematurity, corneal graft rejection, neovascular glaucoma and corneal scaring. Any abnormal growth of blood vessels in the eye can scatter and block the incident light prior to reaching the retina. Neovascularization can occur at almost any site in the eye and significantly alter ocular tissue function. Some of the most threatening ocular neovascular diseases are those which involve the retina. For example, many diabetic patients develop a retinopathy which is characterized by the formation of leaky, new blood vessels on the anterior surface of the retina and in the vitreous causing proliferative vitreoretinopathy. A subset of patients with age related macular degeneration develop subretinal neovascularization which leads to their eventual blindness.

Diabetic Retinopathy occurs when the retinal vessels inside the eye leak blood and fluids into the surrounding tissue. About 80% of patients with diabetes develop diabetic retinopathy. This disease is generally treated using a laser. However, laser therapy involves complications including retinal vein occlusion, loss of visual acuity, vitreous hemorrhage and sometimes failure. If left untreated, diabetic retinopathy may cause blindness.

Retinopathy of Prematurity (ROP) affects prematurely born babies. It consists of the abnormal growth of blood vessels within the retinal and vitreous. Progression to later stages of ROP can lead to the formation of scar tissue on the retina, vitreous hemorrhage, and retinal detachment. The treatment is usually performed either by laser or cryotherapy (freezing).

Ischemic retinopathies are retinopathies associated with vascular occlusion (capillaries or large vessels) that lead to neuroretinal suffering, cell death and neo angiogenesis. Macular degeneration is a disease that affects central vision and leads to loss of vision. Although there are forms of macular degeneration that strike young people, the condition occurs most commonly in people who are over 60 years of age. This disorder is thus called age-related macular degeneration (AMD). Because only the center of a person's vision is usually affected, blindness rarely occurs from the disease. However, injury to the macula in the center of the retina can destroy the ability to see straight ahead clearly. Dry forms associate degeneration of neuroretina, RPE cells and choroids. Wet forms associate previously described phenomenons and growth of neovessels from the choriocapillaries and/or retinal vessels, sub retinal detachment and hemorrhages, sub epithelial hemorrhages and tears, etc. Macular degeneration usually occurs after the age of sixty. While your central vision is reduced, most patients retain some vision and never go totally blind.

A particular aspect of the invention is a method of treating intraocular neovessels or macular oedema comprising delivering to the suprachoroidal space of a subject suffering therefrom a nucleic acid encoding an anti VEGF, an anti VEGF receptor or an anti PLGF.

A further particular aspect of the invention is a method of treating or delaying retinitis pigmentosa comprising delivering to the suprachoroidal space of a subject suffering therefrom a nucleic acid encoding a neurotrophic factor as described above.

Another particular aspect of the invention is a method of treating diabetic retinopathy comprising delivering to the suprachoroidal space of a subject suffering therefrom a nucleic acid encoding a a nucleic acid encoding an anti IRS-1 or IGF-1.

### Operation

To use an electroporation device shown in FIGS. 1 to 4, an operator may proceed by the following steps:

First, the operator couples the Luer cone 32 to the syringe 31 filled with the pharmaceutical product, and electrically connects the first and second connectorsto the two terminals of the electrical generator 6. Preferably, the first connector is connected to the “+” terminal.

To position the injection device on the eye Y, the operator pulls the proximal parts 13 and 13′ apart so that the first and second cups 16 and 16′ be spaced from each other, reaching the “remote” position. Preferably, in the remote position, the first and second cups are spaced from each other by a distance of at least 30 mm, preferably at least 40 mm, and/or less than 80 mm, or less than 60 mm.

The operator may then introduce the second cup 16′ so that the posterior region of the eye be in contact with the second contact surface, and position the locating mark 19 on the limbus L, the first support being in the disengaged position where the injection needles are not penetrating in the eye, and being preferably locked in this disengaged position.

The spring 44 maintains the first and second arms close to each other.

While keeping the second contact surface 18′ in contact with the outside surface of the eye Y and the locating mark 19 bearing on the limbus L, the operator then unlocks and rotates the first support around the axis Z, to cause the injection needles to penetrate through the outside surface of the eye. The injection needles penetrate substantially simultaneously through this surface, until the first contact surface 18 comes into contact with the outside surface of the eye.

The spring 20 maintains the first contact surface in contact with the outside surface of the eye.

The injection device is then in the “close” position, the eye Y being pressed between the first and second contact surfaces, as shown in FIG. 1.

The shape of the second surface, matching the surface of the eye, and the bearing of the locating mark on the limbus advantageously permit a stable but also very precise positioning. The shape of the second contact surface also facilitates the manipulation of the injection device during the stage of penetration of the injection needles.

The shape and arrangement of the injection needles are determined such that, in the close position, the operator is guaranteed that the ejection orifices of the injection needles open into the suprachoroidal space. The operator then knows that the pharmaceutical product will be properly injected into the suprachoroidal space, in a homogeneous manner.

The shape and arrangement of the injection needles are also determined such that, during the injection, no ejection orifice of any injection needle is at less than 4 mm away from the limbus.

The operator then immobilizes the injection device in this position. The bearing of the first and second contact surfaces on the sclera and the insertion of the needles in the eye Y provide good stability of the injection device.

The operator may then begin the injection of the pharmaceutical product by acting on the piston of the syringe 31.

The increase in the local pressure in the area of the injection points is believed to promote the introduction of the injected product into the cells, particularly in the case of transfection. A person skilled in the art therefore generally considers it preferable to limit the number of injection points, if possible by using only a single injection needle. Surprisingly, however, the inventors have found that multiplication of the injection points promotes the penetration of the product.

The operator then sends a suitable electrical signal by means of the electrical generator, in such a way as to create, within the injection zone, an electrical field that promotes electroporation. The arrangement and the shapes of the first and second electrodes, in this case the electrically conductive first and second contact surfaces, make it possible to create an electrical field particularly effective for electroporation.

In a particular embodiment, an electrical field constituted by one or more electrical pulse(s) is applied.

The field intensity of which is preferably between about 1 and 600 Volts, preferably 1 and 400 Volts, even more preferably between about 1 and 200 Volts, advantageously between about 10 and 100 Volts, or 15 and 70 Volts.

The total duration of application of the electric field may be between 0.01 millisecond and 1 second, preferably between 0.01 and 500 milliseconds, more preferably between 1 and 500 milliseconds, even more preferably greater than 1 or 10 milliseconds. In a preferred embodiment, the total duration of application of the electric field is between 10 milliseconds and 100 milliseconds and is preferably of 20 milliseconds.

The number of electric pulses applied may be between for example 1 and 100 000. Their frequency may be comprised between 0.1 and 1000 hertz. It is preferably a regular frequency.

Electric pulses may also be delivered in an irregular manner relative to each other, the function describing the intensity of the electric field as a function of the time for one pulse being preferably variable.

Electric pulses may be unipolar or bipolar wave pulses. They may be selected for example from square wave pulses, exponentially decreasing wave pulses, oscillating unipolar wave pulses of limited duration, oscillating bipolar wave pulses of limited duration, or other wave forms. Preferentially, electric pulses comprise square wave pulses or oscillating bipolar wave pulses.

When the electroporation of the pharmaceutical product has been completed, the operator electrically disconnects the electrodes and the generator and then moves the first support into the disengaged position, so as to withdraw the injection needles from the eye. He then moves the first and second cups 16 and 16′ away from each other and removes the injection device.

As will now be clear, the injection device according to the invention permits
precise and stable positioning on the outside surface of the eye of the electrodes and of the injection needles, before penetration of the injection needles;
precise guidance of the injection needles during their penetration into the eye;
precise injection into the suprachoroidal space;
provision of large first and second electrodes; and
high stability of the first and second electrodes during the electroporation, in a position enabling the generation of an efficient large electrical field, exactly where the pharmaceutical product has been injected.

Of course, the invention is not limited to the embodiments described and shown, which have been provided by way of illustration.

In particular, the shape of an injection needle is not limited. The injection needle may be substantially rectilinear. It may also extend along an arc of a circle, in particular in order to facilitate its insertion by rotation of the first support around the axis Z.

The injection needles may be parallel or not. But it is preferable that they are substantially parallel.

The injection needles may have an identical length, whichever injection needle is considered. They may also have different length, provided that, when the first contact surface is in contact with the sclera, the pharmaceutical composition is injected within the suprachoroidal space.

Moreover, the movement of the first electrode relative to the second electrode may be guided differently. For instance, it could be guided so that the first electrode translates between the remote position and the close position.

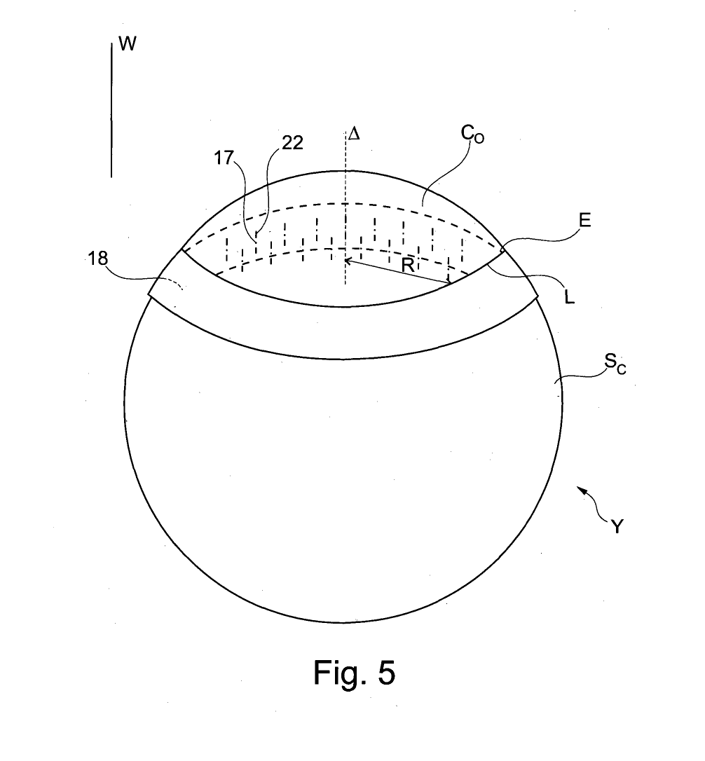

Also, in one embodiment, as represented in FIG. 5, the first contact surface 18 may be perforated with a single hole 18′. One or several characteristics of the other embodiments described here above may be applied to this embodiment.

Preferably the hole 18′ is located in the center of the first contact surface. The hole 18′ is preferably circular, the radius R of the hole being preferably greater than 5 mm or greater than 6 mm and/or less than 8 mm or less than 7 mm, a radius of 6.58 mm being preferred.

Preferably the edge E of said hole is designed to be placed in contact with the surface of an eye, in particular to bear on the edge of the cornea Co, i.e. the limbus L. This edge can in particular be formed by a band of flexible material with a width of greater than 1.5 mm and/or less than 5 mm. It can in particular be formed by a bead of silicone or of foam. Advantageously, the risk of injury to the limbus is thereby reduced.

Preferably, the injection needles 17 defines an angle comprised between 45° and 90° with the surface of the first contact surface. Preferably, they are inserted perpendicularly to the surface of the first contact surface.

In an embodiment, the injection needles 17 all extend parallel to each other, preferably parallel to the axis Δ of the hole 18′.

As represented in FIG. 5, the first contact surface may have the shape of a ring, or of a portion of a ring.

Said ring or said portion of a ring has preferably a width greater than 3 mm and less than 8 mm.

When the first contact surface has a hole 18′, or has the shape of a ring or of a portion of a ring extending on more than 180°, the length of the injection needles is preferably less than 1.5 mm, preferably less than 1.0 mm, preferably less than 900 μm and/or greater than 500 μm, or greater than 700 μm, the length of the injection needles being preferably 800 μm.

When the first contact surface has a hole 18′, or has the shape of a ring or of a portion of a ring extending on more than 180°, the device preferably comprises more than 5, more than 7, and/or less than 30, less than 20, less than 15 injection needles, the number of injection needles being preferably 10.

1. An injection device comprising:
a first support having a cup-shaped first contact surface intended to come into contact with a first region of an outside surface of an eye,
a set of at least four injection needles in fluid communication with each other and protruding from said first contact surface at respective insertion points so that the distance between the distal end of any of said injection needles to said first contact surface is between 0.6 mm and 1.3 mm,
the insertion points of the injection needles on the first contact surface being spread on said first contact surface so that the diameter of the largest circle that it is possible to include completely in the convex surface defined by said insertion points on a front view of said first contact surface, without any insertion point being included in said circle, is less than 12 mm, the injection device comprising a first electrode designed to be connected electrically to a first terminal of an electrical generator, said first electrode comprising at least a part, preferably the whole region of the first support defining the first contact surface.

2. An injection device according to claim 1, wherein the diameter of the largest circle that it is possible to include completely in the convex surface defined by said insertion points on a front view of said first contact surface, without any insertion point being included in said circle, is less than 6 mm.

3. An injection device according to claim 1, wherein the insertion points are spread homogeneously in said convex surface on said front view.

4. An injection device according to claim 1, comprising more than 10 injection needles.

5. An injection device according to claim 1, wherein the distance between the first contact surface and the distal end of any injection needle is more than 0.7 mm and less than 1.0 mm.

6. An injection device according to claim 1, wherein the shape of said first contact surface is spheroidal or ellipsoidal, the radius of curvature at any point of the first contact surface being greater than 9 mm and less than 15 mm.

7. An injection device according to claim 1, wherein the first contact surface has a surface area greater than 100 mm².

8. An injection device according to claim 7, comprising a locating mark following an arc of a circle having a radius of greater than 5 mm and of less than 8 mm.

9. An injection device according to claim 8, wherein the distance between any of said insertion points and said locating mark is greater than 4 mm.

10. An injection device according to claim 9, wherein the injection needles substantially extend along a common general direction, the injection device comprising a first proximal part, the first support being rotationally mounted on said first proximal part around an axis substantially perpendicular to said direction.

11. An injection device according to claim 10, wherein the first support may rotate between a disengaged position where the injection needles are not penetrating in the eye and an engaged position where the injection needles are introduced in the eye, while allowing a locating mark remaining in contact with the limbus of the eye during the rotation between these two positions.

12. An injection device according to claim 1, said first electrode comprising one, preferably several, preferably all the injection needles.

13. An injection device according to claim 1, comprising a second electrode designed to be connected electrically to a second terminal of said electrical generator and mobile relative to the first electrode between a close position and a remote position in which the second electrode is close to and remote from the first electrode, respectively, the second electrode being guided during the movement between the remote position and the close position.

14. An injection device according to claim 13, comprising first and second arms supporting said first and second electrodes, respectively, the movement of the second arm being guided relative to the first arm, preferably the second arm being rotationally mounted on the first arm.

15. An injection device according to claim 14, wherein the second electrode comprises a cup-shaped electrically conductive second contact surface configured so as to be in contact, in the close position, with a second region of said outside surface of the eye, preferably so as to match said second region of said outside surface, the second region being opposite to the first region.

16. An injection device according to claim 15, wherein the first contact surface is perforated with a single hole.

17. An injection device according to claim 16, wherein said single hole is circular and presents a radius greater than 5 mm and less than 8 mm.

18. Method for injecting a composition into the suprachoroidal space of an eye by means of an electroporation device comprising
an injection device according to any one of the preceding claims, and
an electrical generator having first and second terminals,
the set of injection needles and/or the first contact surface of said injection device being electrically connected to said first terminal, said method comprising the following steps:
a) inserting the injection needles into the eye until the first contact surface comes into contact with a first region of the outside surface of the eye, the injection needles being configured so that, in this position, they open out into said suprachoroidal space,
b) injecting said composition through said injection needles,
c) independently of the preceding steps, applying a counter electrode, preferably a second electrode of the injection device, on a second region of the outside surface of the eye, the second region being substantially opposite to the first region relative to the centre of the eye,
d) independently of the preceding steps, connecting the first electrode and the counter electrode to said first and second terminals, respectively,
e) generating an electrical field between said first and counter electrodes with said electrical generator, the electrical field being adapted to promote electroporation.
